(19) United States
(12) Patent Application Publication
(10) Pub. No.: US 2014/0114261 A1
(43) Pub. Date: Apr. 24, 2014

(54) METHOD AND DEVICE FOR DISPENSING A LIQUID FROM A COLLAPSIBLE CONTAINER

(71) Applicant: NORDSON CORPORATION, Westlake, OH (US)

(72) Inventors: Kevin C. Geppert, Eagan, MN (US); Todd W. Sharratt, Stillwater, MN (US)

(73) Assignee: NORDSON CORPORATION, Westlake, OH (US)

(21) Appl. No.: 13/777,522

(22) Filed: Feb. 26, 2013

Related U.S. Application Data
(60) Provisional application No. 61/717,340, filed on Oct. 23, 2012.

(51) Int. Cl. A61M 11/00

(52) U.S. Cl. CPC A61M 11/00
USPC 604/290; 604/310

(57) ABSTRACT

A method and device for dispensing a liquid from a tip includes a support structure, a cartridge, and a drive unit. The support structure is adapted for supporting a collapsible container having a connector end and a closed end. The support structure includes a liquid passage adapted for fluid communication with the collapsible container for selectively discharging the liquid. The cartridge includes an actuator selectively movable from a first position to the second position via the operatively connected drive unit. At least a portion of the actuator moves toward the connector end when moving from the first position to the second position. The actuator is adapted for gradually compressing the collapsible container from the closed end toward the connector end in order to discharge liquid droplets.

### CROSS-REFERENCE TO RELATED APPLICATION

This application claims the priority of U.S. Provisional Patent Application Ser. No. 61/717,340, filed on Oct. 23, 2012 (pending), the disclosure of which is incorporated by reference herein.

### TECHNICAL FIELD

The present invention relates generally to a method and device for dispensing a liquid, and more particularly, to a method and device for dispensing reactive liquids for use in a medical procedure.

### BACKGROUND

Generally, it is known to dispense reactive liquids in the form of sprayed droplets for use in various fields. For example, a plurality of reactive liquids, such as biomaterials, may be sprayed to disperse and dispense the droplets on the human body or within the human body during a medical procedure. For instance, two highly reactive fluids may be sprayed onto an anatomical site for reducing the flow of blood by hemostatic clotting or creating tissue barriers to prevent anatomical tissues from adhering together during and/or after the medical procedure. Ideally, these reactive liquids are isolated prior to being discharged. The reactive liquids mix and react once discharged at the anatomical site.

Reactive liquids used in medical procedures are frequently stored within vials. Vials are generally rigid, hollow structures, such as glass tubes, having a plug slidably inserted into an open end of the vial. The vials are removably connected to an applicator. The applicator moves the plug into the vial in a piston-like manner. This forces the reactive liquid from the vial into a passage and a fluidly connected tip for selectively dispensing the reactive liquid.

While the vial serves as a sufficient container for storing the reactive liquid, the rigidity of the vial limits its adaptability with various applicators. For example, traditional applicators include a plunger to force the plug into the vial for dispensing the reactive liquid. However, the plunger must be generally as long as the vial in order to force the plug along the length of the vial. The necessity for both the plunger and the vial nearly doubles the required length for the applicator. Thus, applicators connected to rigid vials are generally limited to shapes and sizes that may be otherwise inconvenient for use during the medical procedure.

There is a need for a method and device for use in dispensing a liquid, such as during a medical procedure, that addresses present challenges and characteristics such as those discussed above.

### SUMMARY

One exemplary embodiment of the invention is a method for dispensing a liquid from a collapsible container from a device having a cannula communicating with a tip and a cartridge that has an actuator. The method includes moving at least a portion of the actuator of the cartridge from a first position to a second position to compress the collapsible container. In addition, the method includes compressing the collapsible container from a closed end of the collapsible container toward a connector end of the collapsible container. The liquid discharges from the collapsible container to fluidly communicate the liquid along the cannula and to the tip. Accordingly, the method further includes dispensing the liquid from the tip connected to the cannula. As the liquid dispenses, the liquid forms into droplets of liquid for use during the medical procedure.

In one aspect, the actuator includes a compression member and a slide member. The method includes sliding the slide member in a slot to engage the compression member. As the compression member is engaged, the slide member moves the compression member from the first position to the second position to compress the collapsible container.

In another aspect, the actuator includes a compression member having a compression end. The method includes pivoting the compression member from the first position to the second position. As the compression member pivots, the collapsible container is compressed between the compression end and the compression surface.

Another exemplary embodiment of the invention is a method for dispensing two reactive liquids from two collapsible containers, respectively. The liquids are dispensed from a device having a cannula communicating with a tip and a cartridge that has an actuator. The method includes moving at least a portion of the actuator of the cartridge from a first position to a second position to compress each of the collapsible containers from a closed end toward a connector end. The method further includes discharging the reactive liquids to fluidly communicate the reactive liquids to the tip and dispensing the reactive liquids from the tip. Accordingly, the reactive liquids form into droplets of mixed reactive liquids for use in the medical procedure.

In addition, the two reactive liquids are two biomaterials. The method also includes applying droplets of the mixed biomaterials to an anatomical site during the medical procedure. In one aspect, the actuator includes a compression member having a slide member. The method includes sliding the slide member to engage the compression member and moving the compression member from the first position to the second position. In another aspect, the actuator includes a compression member. The method includes pivoting the compression member from the first position to the second position.

Another exemplary embodiment of the invention is a device that dispenses a liquid from a tip. The liquid is held within a collapsible container having a connector end and a closed end. The device generally includes a support structure adapted to support the collapsible container, a cartridge, and a drive unit. The support structure has a liquid passage adapted for fluidly communicating discharged liquid from the cannula. The liquid dispenses from the tip forming droplets of the liquid for appropriate application. The cartridge is connected to the support structure and has an actuator selectively movable from a first position to a second position. At least a portion of the actuator moves toward the connector end that is fluidly connected to the liquid passage when moving from the first position to the second position. The drive unit is also connected to the support structure and operatively connected to the cartridge. The drive unit is adapted to move the actuator from the first and second position for gradually compressing the collapsible container from the closed end toward the connector end. The movement of the actuator from the first position to the second position discharges the liquid from the liquid passage.

In one embodiment of the cartridge, the cartridge includes a compression surface and a pair of sidewalls. The actuator includes a compression member and a slide member and extends between the pair of sidewalls for moving to compress the collapsible container against the compression surface. The compression member is pivotably mounted to a cartridge body for moving from the first position to the second position. The slide member is supported by the cartridge body to slide longitudinally along the cartridge body to engage the compression member and force the compression member to pivot from the first position to the second position. Accordingly, liquid moves toward the tip for being dispensed in order to form droplets of liquid.

In another embodiment of the cartridge, a cartridge body is generally cylindrical and includes an outer wall with a compression surface and an inner wall. The outer and inner walls define a generally cylindrical passage therebetween. Generally, the cartridge body is adapted to support the collapsible container within the generally cylindrical passage adjacent to the compression surface. More particularly, the generally cylindrical cartridge body has a central axis. The actuator includes a compression end and is pivotably mounted about the central axis for moving from the first position to the second position. The actuator extends through the inner wall toward the outer wall to compress the collapsible container against the compression surface and move the liquid toward the tip for being dispensed in order to form droplets of liquid.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description given below serve to explain the invention.

FIG. 1 is a perspective view of a device according to a first embodiment of the invention.

FIG. 2 is a perspective view of a cartridge according to the first embodiment of the device shown in FIG. 1.

FIG. 3A is a cross-sectional view of FIG. 2 taken along section line 3-3.

FIG. 3B is another cross-sectional view of FIG. 2 taken along section line 3-3.

FIG. 4 is a perspective view of a device according to a second embodiment of the invention.

FIG. 5 is a perspective view of a cartridge according to the second embodiment of the device shown in FIG. 4.

FIG. 6A is a cross-sectional view of FIG. 5 taken along section line 6-6 with the actuator in a first illustrative position.

FIG. 6B is a cross-sectional view similar to FIG. 6A but showing the actuator in another position.

### DETAILED DESCRIPTION

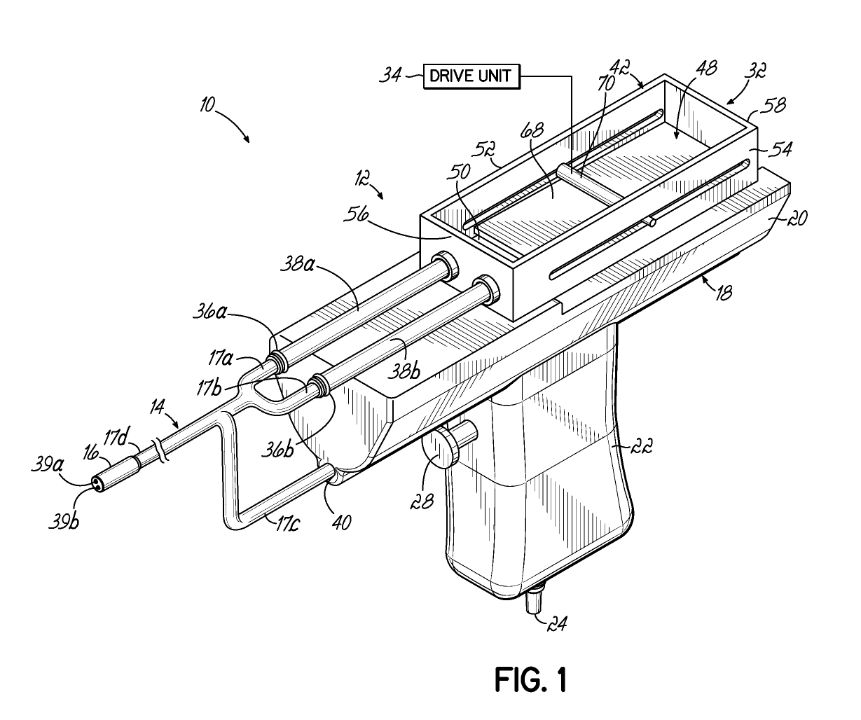

FIG. 1 shows a first embodiment of a device 10 for dispensing liquids, such as reactive liquids, and a gas with an applicator 12, particularly for use during medical procedures. A cannula 14 is connected to both the applicator 12 and a tip 16 configured for dispensing the liquids and gas. More particularly, the cannula 14 includes a pair of proximal ends 17a, 17b, a gas supply end 17c, and a distal end 17d. The distal end 17d is connected to the tip 16 while the proximal ends 17a, 17b and the gas supply end 17c are connected to the applicator 12. The cannula 14 may be rigid, flexible, or flexible and steerable. The cannula 14 is adapted to deliver two liquids and the gas separately to the tip 16 for spraying droplets during applications of any desired type, e.g., medical procedures. The liquids mix while exiting the tip 16 and form into droplets under the influence of the adjacently dispensed gas. One such tip 16 that may be used with the applicator 12, or other applicators within the scope of this disclosure, is the low-pressure tip of U.S. Provisional Patent Application Ser. No. 61/681,760, the disclosure of which is incorporated herein by reference in its entirety. However, it will be appreciated that the reactive liquids may be dispensed without the aid of gas. Thus, the device is not intended to be limited to the applicator 12 having gas related features. Generally, any tip, cannula, or other component(s) for dispensing liquid with or without gas may be used. For example, one such component used for dispensing liquid droplets without using gas assist is the Rigid Dual Cannula System of the FibriJet® Tips product line produced by Nordson® Micromedics®. Another such component is the sprayer assembly described within U.S. Pat. No. 5,368,563. The medical procedures for use with the device 10 may include topical applications, open surgical applications, and minimally invasive applications such as laparoscopy. Furthermore, reactive liquids used during such medical procedures may be biomaterials that include, but are not limited to, fibrin and thrombin for coagulating blood during the medical procedure.

The applicator 12 generally includes a support structure 18 having a body 20 and a handle 22 for an operator to grip by hand. The handle 22 includes a gas supply conduit 24 for being operatively connected to a pressurized gas supply (not shown). The handle 22 also supports a control unit such as a trigger 28 for operatively and selectively dispensing at least liquid from the tip 16. The gas may also be dispensed through the operation of the trigger 28 or any other suitable control unit. For example, one alternative control unit is a foot switch. According to the exemplary embodiment, the trigger 28 is configured to be manipulated by a finger or fingers of the operator while gripping the handle 22 by hand. The trigger 28 is biased such that by applying force toward the handle 22, the operator may selectively withdraw the trigger 28 toward the handle 22 for operating the applicator 12. While the trigger 28 is movable for selectively operating the applicator 12, it will be appreciated that any known method of controls for providing user input may be used for operating the applicator 12. For example, rather than moving the trigger 28, the control unit may accept user input via one or more mechanical switches, electrical switches, or computer interface for dispensing the gas and the liquid.

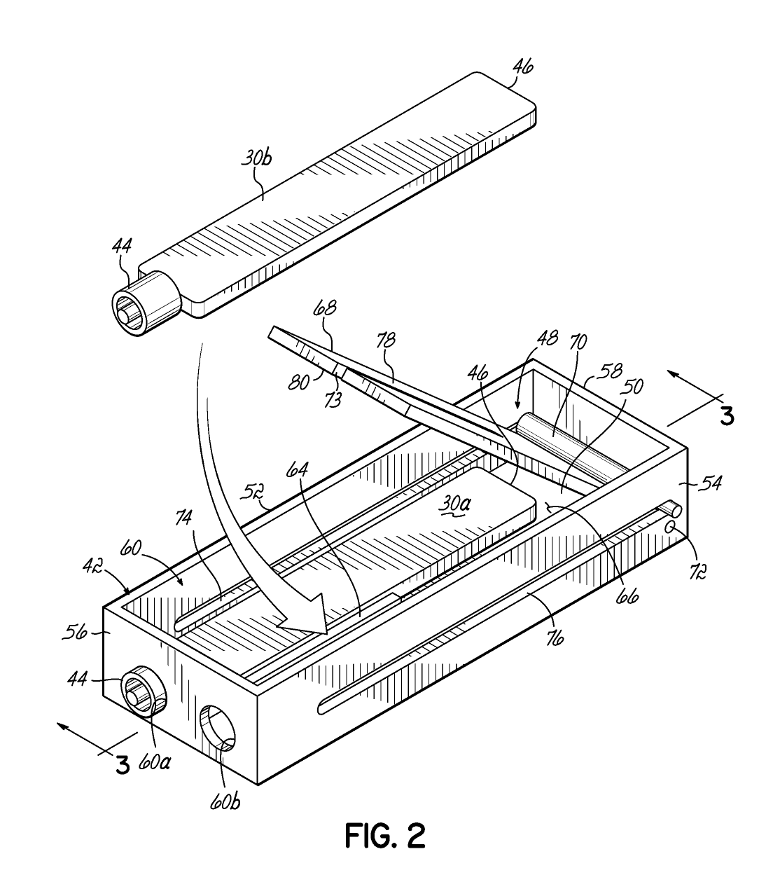

With respect to FIGS. 1 and 2, the body 20 is adapted to support collapsible containers 30a, 30b within a cartridge 32 for holding respective liquids. Alternatively, the cartridge 32 may hold just a single container of liquid, or more than two containers. According to the exemplary embodiment, the cartridge 32 is directly connected to the body 20; however, the cartridge 32 may, in the alternative, be either indirectly connected to the body 20 or removably connected to the body 20. Because the cartridge 32 of the exemplary embodiment of the device 10 is fixed to the body 20, collapsible containers 30a, 30b are removably loaded into the cartridge 32 and replaced when empty of liquid to refill the applicator 12. Alternatively, the cartridge 32 may be removably connected to the body 20 having collapsible containers 30a, 30b preloaded into the cartridge 32. Accordingly, as the collapsible containers 30a, 30b empty during use, the entire cartridge 32 may be replaced to refill the applicator 12.

A drive unit 34 is connected to the cartridge 32. It will be appreciated that any drive unit 34, such as mechanical, electrical, pneumatic, or any combination thereof, may be used to operatively drive the cartridge 32 to expel liquid from the collapsible containers 30a, 30b. The drive unit 34 is operatively connected to the trigger 28 for selectively dispensing the liquid from the tip 16. According to the exemplary embodiment, the body 20 further includes a pair of coupling ends 36a, 36b having liquid passages 38a, 38b extending therethrough. The liquid passages 38a, 38b each are adapted to fluidly communicate with the collapsible containers 30a, 30b. The coupling ends 36a, 36b each respectively connect to the proximal ends 17a, 17b of the cannula 14 for fluidly communicating the liquids from the collapsible containers 30a, 30b, and into the cannula 14. Accordingly, the drive unit 34 selectively drives the cartridge 32 to compress the collapsible containers 30a, 30b to create enough pressure within the collapsible containers 30a, 30b for discharging the liquid from the liquid passages 38a, 38b and into the proximal ends 17a, 17b. The liquids are maintained within separate lumens (not shown) while moving toward the distal end 17d for dispensing from the tip 16. The tip 16 includes outlets 39a, 39b in respective fluid communication with the proximal ends 17a, 17b. Thus, under pressure, liquid from the collapsible container 30a dispenses from the outlet 39a, while the liquid from the collapsible container 30b dispenses from the outlet 39b. The exemplary embodiment of the device 10 is configured to dispense two liquids; however, it will be appreciated that the device 10 may be similarly configured for dispensing any number of liquids for use in a medical procedure.

The body 20 also includes a gas passage 40 in fluid communication with the gas supply end 17c of the cannula 14 for dispensing gas from the tip 16. The gas passage 40 is in selective fluid communication with the gas supply conduit 24 and operatively connected to the trigger 28. Accordingly, the operator, via the trigger 28, selectively directs gas from the gas supply conduit 24 to the gas passage 40 for discharging gas from the applicator 12 and into the gas supply end 17c. The gas moves along the cannula 14 separated from the liquids while moving toward the distal end 17d. At the tip 16, the gas is dispensed from the tip 16 at both the outlets 39a, 39b in conjunction with the liquids. As such, the liquids mix and form into droplets under the influence of the pressurized gas. According to the exemplary embodiment, the support structure 18 is a unitary, handheld structure having the body 20 directly connected to the handle 22. However, it will be appreciated that the support structure 18 may, in the alternative, be formed of any number of independent support structures that are operatively connected for supporting the device 10. For example, separate mechanical structures may each independently support the collapsible containers 30a, 30b, the drive unit 34, and the trigger 28 such that the device 10, rather than being a handheld unit, is a system of components capable of similarly dispensing the liquid and the gas from the tip 16.

FIG. 2 shows the first embodiment of the cartridge 32. The cartridge 32 generally includes a cartridge body 42. The cartridge body 42 is adapted to support collapsible containers 30a, 30b. As briefly described above, liquids dispensed during medical procedures are frequently dispensed in pairs. Accordingly, the exemplary embodiment of the cartridge 32 supports a pair of the collapsible containers 30a, 30b. However, it will be appreciated that the cartridge 32 may be adapted for any number of collapsible containers 30a, 30b. Moreover, each of the collapsible containers 30a, 30b includes a connector end 44 and a closed end 46. The connector end 44 is a luer lock adapted to fluidly connect to either of the liquid passages 38a, 38b (see FIG. 1) for receiving liquid expelled from the collapsible containers 30a, 30b. The connector end 44 may also be connected to a filling station (not shown) for refilling and reusing the collapsible containers 30a, 30b. The closed end 46 is opposite the connector end 44. According to the exemplary embodiment, the collapsible containers 30a, 30b are flexible, bendable bags that may be squeezed, rolled, or otherwise manipulated to selectively vary the volume within each of the collapsible containers 30a, 30b.

An actuator 48 is operatively connected to the cartridge body 42. The actuator 48 is adapted to compress the collapsible containers 30a, 30b and expel the liquid from the connector end 44. The actuator 48 is movable from a first position to a second position and generally movable therebetween. Specifically, the first position of the actuator 48 is adjacent to the closed end 46, and the second position of the actuator 48 is adjacent to the connector end 44. At least a portion of the actuator 48 moves from the closed end 46 toward the connector end 44 while moving from the first position to the second position. In each of the first and second positions, the actuator 48 contacts and compresses the collapsible containers 30a, 30b. As the actuator 48 moves from the first to the second position, the actuator 48 gradually compresses the collapsible containers 30a, 30b from the closed end 46 toward the connector end 44. The actuator 48 may selectively move from the first position toward the second position and into any position therebetween.

Moreover, the cartridge body 42 includes a base 50 on which the collapsible containers 30a, 30b directly rest. The cartridge body 42 further includes walls 52, 54, 56, 58 connected to and surrounding the base 50. More particularly, first and second opposed sidewalls 52, 54 extend upward from the base 50 while front and rear opposed walls 56, 58 similarly extend upward from the base 50. The walls 52, 54, 56, 58 and the base 50 are rigidly connected, forming a rectangular prism having an opening 60 above the base 50. The first and second sidewalls 52, 54 extend in a generally longitudinal direction along the base 50, while the front and rear walls 56, 58 extend generally transverse to the base 50 and first and second sidewalls 52, 54.

The opening 60 is sized to accommodate the collapsible containers 30a, 30b, which are inserted therein and positioned on the base 50. The front wall 56 has first and second connector holes 60a, 60b, through which the connector ends 44 are inserted for fluid communication with the liquid passages 38a, 38b, respectively. The base 50 also includes a divider wall 64 extending upward from a compression surface 66 of the base 50. The divider wall 64 in conjunction with the walls 52, 54, 56, 58 collectively aid in restraining the collapsible containers 30a, 30b against a compression surface 66 of the base 50 while being compressed by the actuator 48.

The actuator 48 is supported above the compression surface 66 by the walls 52, 54, 56, 58. More particularly, the actuator 48 includes a compression member 68 and a slide member 70. The compression member 68 is pivotably mounted for movement between the first and second sidewalls 52, 54 adjacent to the rear wall 58 via a hinge 72. Thus, the compression member 68 may be pivoted from the first position to the second position. The slide member 70 operatively engages the compression member 68 to selectively force the compression member 68 from the first position to the second position. The slide member 70 is operatively connected to the drive unit 34 (see FIG. 1). Thus, the drive unit 34 (see FIG. 1) moves the compression member 68 via the slide member 70. The compression member 68 also includes a channel 73 extending therethrough. The channel 73 is sized and positioned to accommodate the divider wall 64 as the compression member 68 moves into the second position from the first position.

The slide member 70 is supported by the first and second sidewalls 52, 54, which each have a first and second slot 74, 76, respectively. The first and second slot 74, 76 are similarly positioned to extend through and longitudinally along each sidewall 52, 54. The first and second slots 74, 76 are also sized to accommodate the slide member 70 that transversely extends between the first and second slots 74, 76. As such, the slide member 70 is adapted to slide or otherwise move between the front and rear walls 56, 58 to engage the compression member 68. More particularly, the slide member 70 engages the compression member 68 to apply force toward the compression surface 66 for compressing the collapsible containers 30a, 30b. According to the exemplary embodiment, the first and second slots 74, 76 are generally parallel to the compression surface 66. However, the first and second slots 74, 76 may extend at any angle relative to the compression surface 66 to guide the slide member 70 to engage the compression member 68.

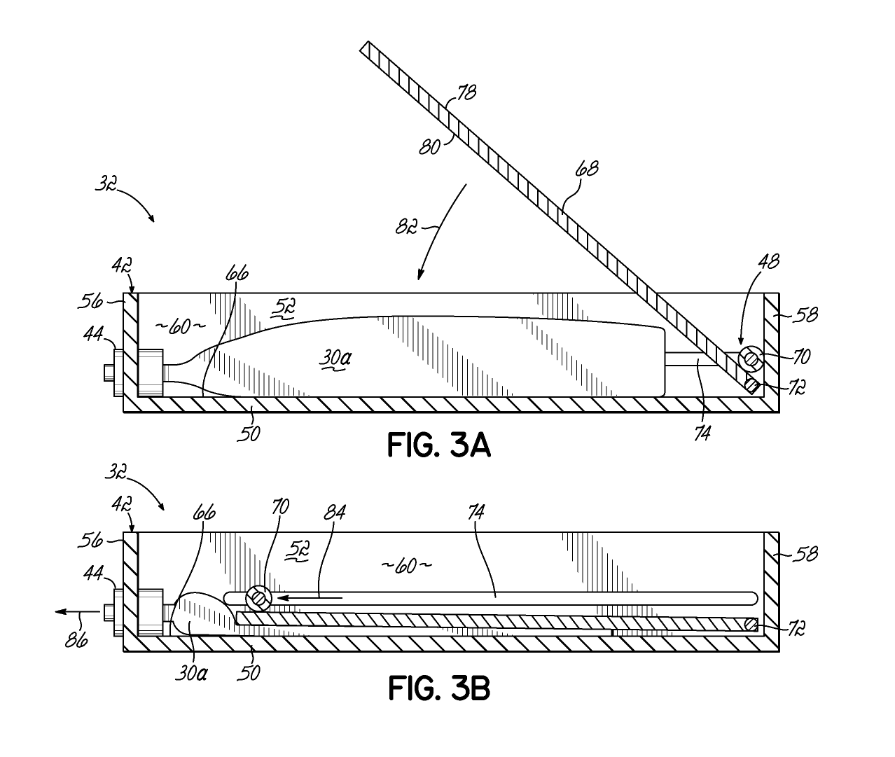

FIG. 3A shows the actuator 48 in the first position. Specifically, the slide member 70 is supported within the first slot 74 directly above the hinge 72 while contacting an upper surface 78 of the compression member 68. Note that the collapsible container 30a shown in FIG. 3A is generally full of the liquid while the compression member 68 is pivoted upward in the first position. A lower surface 80 of the compression member 68 contacts the collapsible container 30a while pivoting from the first position toward the second position as indicated by a first arrow 82.

FIG. 3B shows the actuator 48 pivoted into the second position. Specifically, the slide member 70 is selectively driven generally from the rear wall 58 toward the front wall 56 via the operatively connected drive unit 34 (see FIG. 1) as indicated by a second arrow 84. Accordingly, the slide member 70 engages the compression member 68 to compress the collapsible container 30a from the closed end 46 toward the connector end 44 and expel the liquid as indicated by the third arrow 86. Note that the collapsible container 30a shown in FIG. 3B is generally empty. The actuator 48 may be moved from the first position to the second position at a constant rate or variable rate as selectively directed by the operator. Similarly, the actuator 48 may be selectively stopped in any position between the first position and second position and resumed at any time by the operator. Thus, the rate and timing with which liquid is expelled from the collapsible container 30a may be freely directed by the operator in accordance with the medical procedure.

With respect to FIGS. 1-3B, the applicator 12 is usable by the operator via the trigger 28. Specifically, the operator uses the trigger 28 to selectively dispense gas and/or liquid from the tip 16. Manipulating the trigger 28 fluidly communicates pressurized gas from the gas supply conduit 24 to be selectively dispensed from the tip 16. Further manipulation of the trigger 28 also selectively actuates the drive unit 34. The drive unit 34 selectively drives the actuator 48 of the cartridge 32 for dispensing liquid from the tip 16. More particularly, the collapsible containers 30a, 30b are positioned against the actuator 48. The compression member 68 pivots from the first position to the second position in order to compress the collapsible containers 30a, 30b.

According to the exemplary embodiment of the cartridge 32, the slide member 70 is driven by the drive unit 34 to engage the compression member 68. Thus, the slide member 70 forces the compression member 68 toward the compression surface 66. The collapsible containers 30a, 30b are positioned between the compression surface 66 and the compression member 68 and compressed therebetween as the compression member 68 pivots from the first position to the second position. Notably, the collapsible containers 30a, 30b are compressed from the closed end 46 toward the connector end 44. As the collapsible containers 30a, 30b are compressed, liquid is expelled from the connector end 44, into the liquid passages 38a, 38b, and into the proximal ends 17a, 17b of the cannula 14. Each of the two liquids separately move toward the distal end 17d and into the tip 16 for being dispensed from the outlets 39a, 39b respectively.

Similarly, the gas moves from the gas passage 40 and into the gas supply end 17c. The gas moves along the cannula 14 and into the tip 16. The gas, along with each respective liquids, dispenses from the outlets 39a, 39b. As such, the liquids mix and form into droplets under the influence of the pressurized gas for application onto an anatomical site or other desired location.

Prior to dispensing liquid, the applicator 12 is loaded with one or more collapsible containers 30a, 30b containing the liquid. Once the liquid is expelled from the collapsible containers 30a, 30b, the empty collapsible containers 30a, 30b are removed from the cartridge 32 and full collapsible containers 30a, 30b are loaded into the cartridge 32. In the alternative, the cartridge 32 supporting the collapsible containers 30a, 30b may be removable from the applicator 12. Thus, as the liquid is expelled from the collapsible containers 30a, 30b, the cartridge 32 is removed from the applicator 12 so that another cartridge 32, having full collapsible containers 30a, 30b, are loaded into the applicator 12. In any case, the collapsible containers 30a, 30b may be refilled and reused for providing liquid to the device 10.

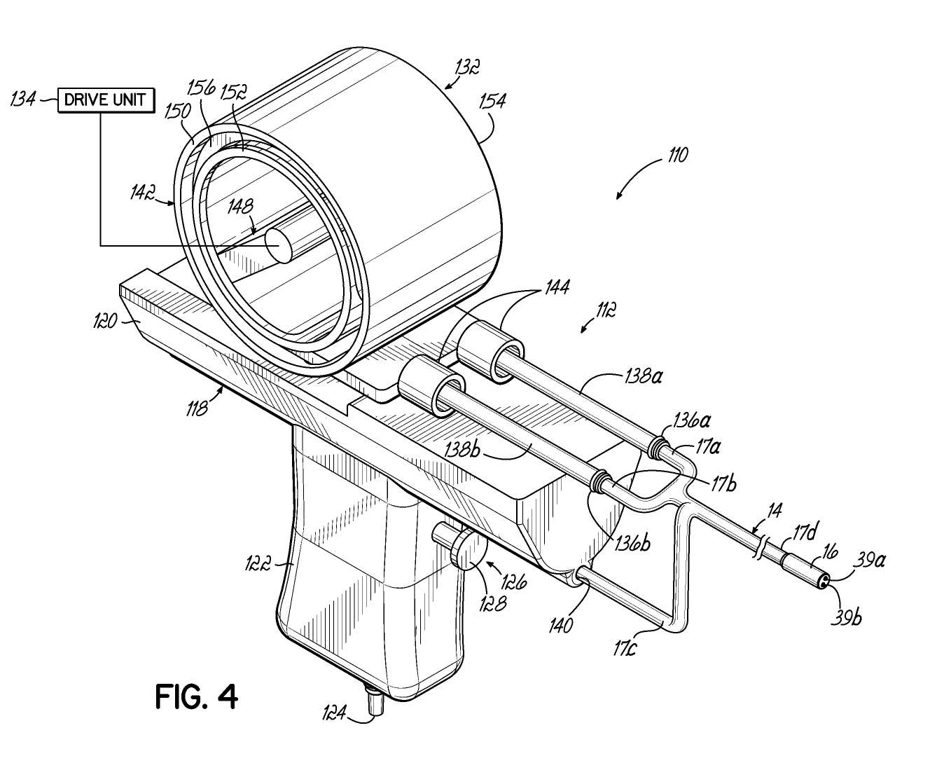

FIG. 4 shows a second embodiment of a device 110 for dispensing liquids, such as reactive liquids, and a gas with an applicator 112, particularly for use during medical procedures. The cannula 14 is connected to both the applicator 112 and the tip 16 configured for dispensing the liquids and gas. More particularly, the cannula 14 includes pair of proximal ends 17a, 17b, a gas supply end 17c, and a distal end 17d. The distal end 17d is connected to the tip 16 while the proximal ends 17a, 17b and the gas supply end 17c are connected to the applicator 12. The cannula 14 may be rigid, flexible, or flexible and steerable. The cannula 14 is adapted to deliver two liquids and the gas separately to the tip 16 for spraying droplets during applications of any desired type, e.g., medical procedures. The liquids mix while exiting the tip 16 and form into droplets under the influence of the adjacently dispensed gas. Such medical procedures for use with the device 110 may include topical applications, open surgical applications, and minimally invasive applications such as laparoscopy.

The applicator 112 generally includes a support structure 118 having a body 120 and a handle 122 for an operator to grip by hand. The handle 122 includes a gas supply conduit 124 for being operatively connected to a pressurized gas supply (not shown). The handle 122 also supports a control unit such as a trigger 128 for operatively and selectively dispensing at least liquid from the tip 16. The gas may also be dispensed through operation of the trigger 128 or another control unit (not shown). For example, the other control unit (not shown) may be operable via a foot switch. According to the exemplary embodiment, trigger 128 is configured to be manipulated by a finger or fingers of the operator while gripping the handle 122 by hand. The trigger 128 is biased from the handle 122 and, by applying force toward the handle 122, the operator may selectively withdraw the trigger 128 toward the handle 122 for operating the applicator 112. While the trigger 128 is movable for selectively operating the applicator 112, it will be appreciated that any known method of controls for providing user input may be used for operating the applicator 112. For example, rather than moving the trigger 128, the control unit may accept user input via one or more mechanical switches, settings, electrical, or computer interface for dispensing the gas and the liquid.

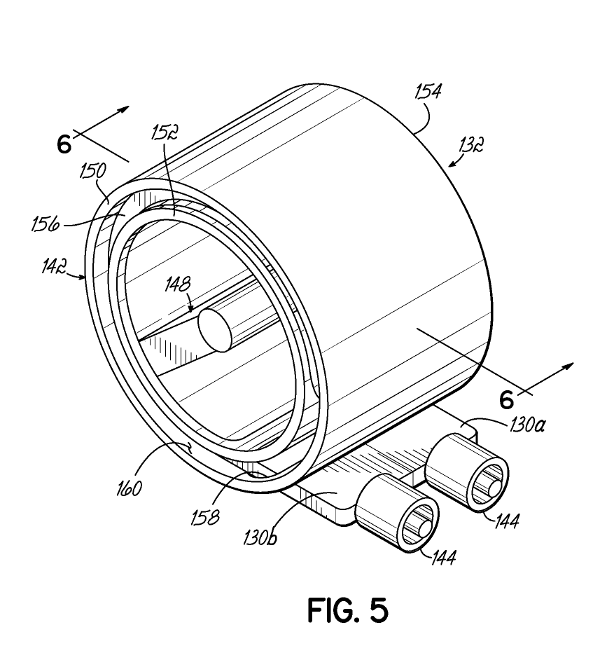

With respect to FIGS. 4 and 5, the body 120 is adapted to support collapsible containers 130a, 130b within a cartridge 132 for holding liquids. Alternatively, the cartridge 132 may hold just a single container of liquid, or more than two containers. According to the exemplary embodiment, the cartridge 132 is directly connected to the body 120; however, the cartridge 132 may, in the alternative, be either indirectly connected to the body 120 or removably connected to the body 120. Because the cartridge 132 of the exemplary embodiment of the device 110 is fixed to the body 120, collapsible containers 130a, 130b are removably loaded into the cartridge 132 and replaced when empty of liquid to refill the applicator 112. Alternatively, the cartridge 132 may be removably connected to the body 120 having collapsible containers 130a, 130b preloaded into the cartridge 132. Accordingly, as the collapsible containers 130a, 130b empty during use, the entire cartridge 132 may be replaced to refill the applicator 112.

A drive unit 134 is connected to the cartridge 132. It will be appreciated that any drive unit 134, such as mechanical, electrical, pneumatic, or any combination thereof, may be used to operatively drive the cartridge 132 to expel liquid from the collapsible containers 130a, 130b. The drive unit 134 is operatively connected to the trigger 128 for selectively dispensing the liquid from the tip 16. According to the exemplary embodiment, the body 120 further includes a pair of coupling ends 136a, 136b having liquid passages 138a, 138b extending therethrough. The liquid passages 138a, 138b each are adapted to fluidly communicate with the collapsible containers 130a, 130b, respectively. The coupling ends 136a, 136b each respectively connect to the proximal ends 17a, 17b of the cannula 14 for fluidly communicating the liquids from the collapsible containers 130a, 130b and into the cannula 14. Accordingly, the drive unit 134 selectively drives the cartridge 132 to compress the collapsible containers 130a, 130b to create enough pressure within the collapsible containers 130a, 130b for discharging the liquid from the liquid passages 138a, 138b and into the proximal ends 17a, 17b. The liquids are maintained within separate lumens (not shown) while moving toward the distal end 17d for dispensing from the tip 16. The tip 16 includes outlets 39a, 39b in respective fluid communication with the proximal ends 17a, 17b. Thus, under pressure, liquid from the collapsible container 30a dispenses from the outlet 39a, while the liquid from the collapsible container 30b dispenses from the outlet 39b. The exemplary embodiment of the device 110 is configured to dispense two liquids; however, it will be appreciated that the device 110 may be similarly configured for dispensing any number of liquids for use in a medical procedure.

The body 120 also includes a gas passage 140 in fluid communication with the gas supply end 17c of the cannula 14 for dispensing from the tip 16. The gas passage 140 is in selective fluid communication with the gas supply conduit 124 and operatively connected to the trigger 128. Accordingly, the operator, via the trigger 128, selectively directs gas from the gas supply conduit 124 to the gas passage 140 for discharging gas from the applicator 112 and into the gas supply end 17c. The gas moves along the cannula 14 separated from the liquids while moving toward the distal end 17d. At the tip 16, the gas is dispensed from the tip 16 at both the outlets 39a, 39b in conjunction with the liquids. As such, the liquids mix and form into droplets under the influence of the pressurized gas. According to the exemplary embodiment, the support structure 118 is a unitary, handheld structure having the body 120 directly connected to the handle 122. However, it will be appreciated that the support structure 118 may, in the alternative, be formed of any number of independent support structures that are operatively connected for supporting the device 110. For example, separate mechanical structures may each independently support the collapsible containers 130a, 130b, the drive unit 134, and the trigger 128 such that the device 110, rather than being a handheld unit, is a system of components capable of similarly dispensing the liquid and the gas from the tip 16.

FIG. 5 shows the second embodiment of the cartridge 132. The cartridge 132 generally includes a cartridge body 142. The cartridge body 142 is adapted to support the collapsible containers 130a, 130b. As briefly described above, the liquids dispensed during the medical procedures are frequently dispensed in pairs. Accordingly, the exemplary embodiment of the cartridge 132 supports a pair of the collapsible containers 130a, 130b connected together in a side-by-side configuration. The collapsible containers 130a, 130b may be formed together but divided internally into the separate collapsible containers 130a, 130b for storing the liquids, or, in the alternative, be individual collapsible containers 30a, 30b (see FIG. 2). However, it will be appreciated that the cartridge 132 may be adapted for any number of collapsible containers 130a, 130b. Moreover, each of the collapsible containers 130a, 130b includes a connector end 144 and a closed end 146. The connector end 144 is a luer lock adapted to fluidly connect to either of the liquid passages 138a, 138b (see FIG. 4) for receiving liquid expelled from the collapsible containers 130a, 30b. The connector end 144 may also be connected to a filling station (not shown) for refilling and reusing the collapsible containers 130a, 130b. The closed end 146 is opposite the connector end 144. According to the exemplary embodiment, the collapsible containers 130a, 130b are flexible, bendable bags that may be squeezed, rolled, or otherwise manipulated to selectively vary the volume within each of the collapsible containers 130a, 130b.

An actuator 148 is operatively connected to the drive unit 134 (see FIG. 4) while being supported by the cartridge body 142. The drive unit 134 drives the actuator 148 to compress the collapsible containers 130a, 130b and expel the liquid from the connector end 144. The actuator 148 is movable from a first position to a second position and generally movable therebetween. Specifically, the first position of the actuator 148 is adjacent to the closed end 146, and the second position of the actuator 148 is adjacent to the connector end 144. At least a portion of the actuator 148 moves from the closed end 146 toward the connector end 144 while moving from the first position to the second position. In each of the first and second positions, the actuator 148 contacts and compresses the collapsible containers 130a, 130b. As the actuator 148 moves from the first to the second position, the actuator 148 gradually compresses the collapsible containers 130a, 130b from the closed end 146 toward the connector end 144. The actuator 148 may be selectively moved from the first position toward the second position and into any position therebetween.

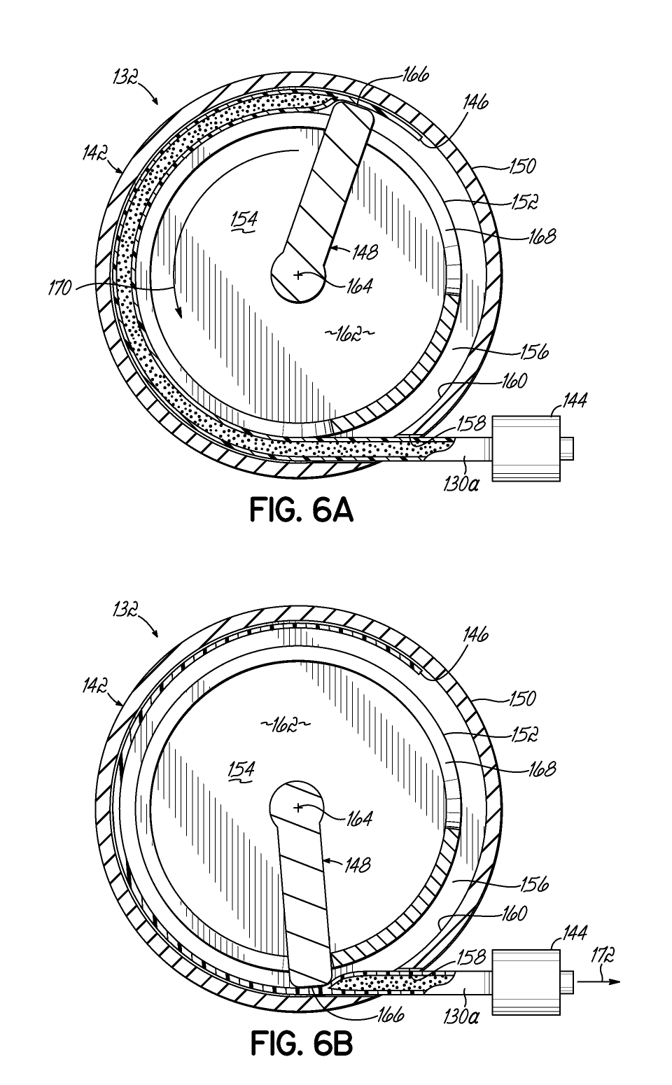

Referring to FIG. 5, as well as FIGS. 6A and 6B, the cartridge body 142 is generally cylindrical and includes an outer wall 150 and an inner wall 152. The outer wall 150 and the inner wall 152 are each connected to a sidewall 154 (see FIG. 6A) in order to space the outer wall 150 from the inner wall 152. Collectively, the outer wall 150 and the inner wall 152 define a generally cylindrical passage 156 in which the collapsible containers 130a, 130b are positioned. The outer wall 150 further includes an opening 158 adapted to receive the collapsible containers 130a, 130b and access the generally cylindrical passage 156. Thus, the cartridge body 142 supports the collapsible containers 130a, 130b within the cylindrical passage 156 and against a compression surface 160 of the outer wall 150.

The cartridge body 142 also includes an inner volume 162 interior of the inner wall 152 defining a central axis 164. The actuator 148 is positioned within the inner volume 162 and adapted to pivot about the central axis 164. More particularly, the actuator 148 is pivotably connected to the sidewall 154 to support the actuator 148 along the central axis 164. The actuator 148 further includes a compression end 166. From the central axis 164, the actuator 148 extends through a channel 168 within the inner wall 152 to the compression end 166. The compression end 166 of the actuator 148 is spaced apart from the compression surface 160 to compress the collapsible containers 130a, 130b therebetween. As briefly described above, the actuator 148 is operatively connected to the drive unit 134 (see FIG. 4) to pivot from the first position to the second position. More particularly, according to the exemplary embodiment, the compression end 166 compresses the collapsible container 130a against the compression surface 160 while the actuator 148 moves from the first position to the second position. Accordingly, the channel 168 is sized to receive the actuator 148 from the first position adjacent to the closed end 146 to the second position adjacent to the connector end 144.

FIG. 6A shows the actuator 148 in the first position. Specifically, the actuator 148 is supported by the sidewall 154 so that the compression end 166 compresses the collapsible containers 130a at the closed end 146. Note that the collapsible container 130a shown in FIG. 3A is generally full of the liquid. The compression end 166 compresses the collapsible bag against the compression surface 160 while pivoting from the first position to the second position, as indicated by a first arrow 170. Thereby, the liquid is forced or otherwise squeezed toward the connector end 144.

FIG. 6B shows the actuator 148 pivoted into the second position. Accordingly, the compression end 166 of the actuator 148 is moved into a position adjacent to the connector end 144 for expelling the liquid, as indicated by the second arrow 172. Note that the collapsible container 130a shown in FIG. 6B is generally empty. The actuator 148 may be moved from the first position to the second position at a constant rate or variable rate as selectively directed by the operator. Similarly, the actuator 148 may be selectively stopped in any position between the first position and second position and resumed at any time by the operator. Thus, the rate and timing with which liquid is expelled from the collapsible container 130a may be freely directed by the operator in accordance with the medical procedure.

With respect to FIGS. 5, 6A, and 6B, the applicator 112 is usable by the operator via the trigger 128. Specifically, the operator manipulates the trigger 128 to selectively dispense gas and/or liquid from the tip 16. Manipulating the trigger 128 fluidly communicates pressurized gas from the gas supply conduit 124 to be selectively dispensed from the tip 16. Further manipulation of the trigger 128 also selectively actuates the drive unit 134. The drive unit 134 selectively drives the actuator 148 of the cartridge 132 for dispensing liquid from the tip 16. More particularly, the collapsible containers 130a, 130b are positioned against the actuator 148. The actuator 148 pivots from the first position to the second position in order to compress the collapsible containers 130a, 130b.

According to the exemplary embodiment of the cartridge 132, the compression end 166 directly contacts the collapsible containers 130a, 130b. The collapsible containers 30a, 30b are positioned between the compression surface 160 and the compression end 166 and compressed between each as the actuator 148 pivots from the first position to the second position. Notably, the collapsible containers 130a, 130b are compressed from the closed end 146 toward the connector end 144. As the collapsible containers 130a, 130b are compressed, liquid is expelled from the connector end 144, into the liquid passages 38a, 38b, and into the proximal ends 17a, 17b of the cannula 14. Each of the two liquids separately move toward the distal end 17d and into the tip 16 for being dispensed from the outlets 39a, 39b respectively.

Similarly, the gas moves from the gas passage 140 and into the gas supply end 17c. The gas moves along the cannula 14 and into the tip 16. The gas, along with each respective liquid, dispenses from the outlets 39a, 39b. As such, the liquids mix and form into droplets under the influence of the pressurized gas for application onto an anatomical site or other appropriate application.

Prior to dispensing liquid, the applicator 112 is loaded with one or more collapsible containers 130a, 130b containing the liquid. Once the liquid is expelled from the collapsible containers 130a, 130b, the empty collapsible containers 130a, 130b are removed from the cartridge 132 and full collapsible containers 130a, 130b are loaded into the cartridge 132. In the alternative, the cartridge 132 supporting the collapsible containers 130a, 130b may be removable from the applicator 112. Thus, as the liquid is expelled from the collapsible containers 130a, 130b, the cartridge 132 is removed from the applicator 112 so that another cartridge 132, having full collapsible containers 130a, 130b, is loaded into the applicator 112. In any case, the collapsible containers 130a, 130b may be refilled and reused for providing liquid to the device 110.

While the present invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative method and device and illustrative examples shown and described. Accordingly, departures may be from such details without departing from the scope or spirit of the general inventive concept.

What is claimed is:

1. A method for dispensing a liquid within a collapsible container from a device during a medical procedure, the device including a cannula communicating with a tip and a cartridge having an actuator, the method comprising;
moving at least a portion of the actuator from a first position to a second position to compress the collapsible container from a closed end of the collapsible container toward a connector end of the collapsible container;
discharging the liquid from the collapsible container to fluidly communicate the liquid along the cannula and to the tip connected thereto;
dispensing the liquid from the tip; and
forming droplets of liquid for use during the medical procedure.

2. The method of claim 1 further comprising dispensing a gas from the tip and spraying the liquid with the gas to form droplets of liquid.

3. The method of claim 1 wherein the device includes an applicator with a control unit operatively connected to the actuator, the method further comprising selectively manipulating the control unit to move the actuator and dispense the liquid.

4. The method of claim 1 wherein the actuator includes a compression member and a slide member, the method further comprising engaging the compression member with the slide member to move the compression member from the first position to the second position to compress the collapsible container.

5. The method of claim 4 wherein the slide member slides within a slot to engage the compression member.

6. The method of claim 1 wherein the actuator includes a compression member, the method further comprising pivoting the compression member from the first position to the second position to compress the collapsible container.

7. The method of claim 6 wherein the compression member includes a compression end, the method further comprising compressing the collapsible container between the compression end and a compression surface.

8. A method of dispensing two reactive liquids within two collapsible containers, respectively, from a device during a medical procedure, the device including a cannula communicating with a tip and a cartridge having an actuator, the method comprising;
moving at least a portion of the actuator from a first position to a second position to compress the collapsible containers from a closed end of each collapsible container toward a connector end of each collapsible container;
discharging the reactive liquids from the collapsible containers to fluidly communicate the liquid along the cannula and to the tip connected thereto;
dispensing the reactive liquids from the tip; and
forming droplets of mixed reactive liquids for use during the medical procedure.

9. The method of claim 8 further comprising dispensing a gas from the tip and spraying the reactive liquids with the gas to form droplets of mixed reactive liquid.

10. The method of claim 8 wherein the two reactive liquids are two biomaterials, the method further comprising applying the droplets of mixed biomaterials onto an anatomical site during the medical procedure.

11. The method of claim 8 wherein the device includes an applicator with a control unit operatively connected to the actuator, the method further comprising selectively manipulating the control unit to move the actuator and dispense the reactive liquids.

12. The method of claim 8 wherein the actuator includes a compression member and a slide member, the method further comprising engaging the compression member with the slide member to move the compression member from the first position to the second position to compress each of the collapsible containers.

13. The method of claim 12 wherein the slide member slides within a slot to engage the compression member.

14. The method of claim 8 wherein the actuator includes a compression member, the method further comprising pivoting the compression member from the first position to the second position to compress each of the collapsible containers.

15. The method of claim 14 wherein the compression member includes a compression end, the method further comprising compressing each of the collapsible containers between the compression end and a compression surface.

16. A device for dispensing a liquid from a tip for forming droplets of the liquid, the liquid held within a collapsible container having a connector end and a closed end, comprising;
a support structure adapted for supporting the collapsible container, the support structure having a liquid passage, the liquid passage adapted for fluid communication with the collapsible container for selectively discharging the liquid from the liquid passage;
a cartridge connected to the support structure, the cartridge having an actuator selectively movable from a first position to a second position such that at least a portion of the actuator moves toward the connector end when moving from the first position to the second position; and
a drive unit connected to the support structure, the drive unit operatively connected to the cartridge to move the actuator between the first position and the second position, the movement of the actuator from the first position to the second position being adapted for gradually compressing the collapsible container from the closed end toward the connector end to discharge the liquid from the liquid passage.

17. The device of claim 16 wherein the liquid is dispensed with a gas from the tip, the support structure further having a gas passage and a gas supply conduit, the gas supply conduit being in selective fluid communication with the gas passage for selectively discharging the gas from the gas passage and spraying the liquid with the gas to form the droplets of the liquid.

18. The device of claim 16 further comprising a cannula in fluid communication with the liquid passage, the cannula having a distal end and a proximal end, the proximal end connected to the liquid passage, the distal end having the tip attached thereto, the tip configured for dispensing the liquid.

19. The device of claim 16 wherein the cartridge further includes a cartridge body, the cartridge body having a compression surface and a pair of sidewalls, the actuator extending between the pair of sidewalls and movable to compress the collapsible container against the compression surface.

20. The device of claim 16 wherein the cartridge further includes a cartridge body and the actuator further includes a compression member, the compression member pivotably mounted to the cartridge body for movement from the first to the second position.

21. A cartridge for use with an applicator configured to dispense a liquid from a collapsible container, the collapsible container having a connector end and a closed end, comprising;
a cartridge body adapted for supporting the collapsible container and configured for being removably attached to the applicator; and
an actuator operatively connected to the cartridge body, the actuator being movable from a first position to a second position such that at least a portion of the actuator moves toward the connector end when moving from the first position to the second position, the movement of the actuator from the first position to the second position being adapted for gradually compressing the collapsible container from the closed end toward the connector end to discharge the liquid from the connector end of the collapsible container.

22. The cartridge of claim 21 wherein the cartridge body further includes a compression surface and a pair of sidewalls, the actuator extending between the pair of sidewalls and movable to compress the collapsible container against the compression surface.

23. The cartridge of claim 21 wherein the actuator further includes a compression member pivotably mounted to the cartridge body for movement from the first position to the second position.

24. The cartridge of claim 23 wherein the actuator further includes a slide member supported by the cartridge body to slide longitudinally along the cartridge body, the slide member sliding to engage the compression member and force the compression member to pivot from the first position to the second position.

25. The cartridge of claim 24 wherein the cartridge body further includes a pair of sidewalls, each of the sidewalls having a slot extending longitudinally along the cartridge body, the slide member adapted to slide within the slots.

26. The cartridge of claim 21 wherein the cartridge body is generally cylindrical and includes an outer wall having a compression surface and an inner wall, the outer and inner walls defining a generally cylindrical passage therebetween, the cartridge body adapted to support the collapsible container within the generally cylindrical passage adjacent the compression surface.

27. The cartridge of claim 26 wherein the outer wall has an opening for receiving the collapsible container.

28. The cartridge of claim 26 wherein the generally cylindrical cartridge body has a central axis, the actuator being pivotably mounted for movement about the central axis between the first and second positions and extending through the inner wall toward the outer wall.

29. The cartridge of claim 28 wherein the actuator has a compression end for compressing the collapsible container against the compression surface.
